(19) United States
(12) Patent Application Publication
(10) Pub. No.: US 2022/0015853 A1
(43) Pub. Date: Jan. 20, 2022

(54) SURGICAL SLEEVE

(71) Applicant: Arete Innovation LLC, Larkspur, CO (US)

(72) Inventor: Jeremy Hedges, Larkspur, CO (US)

(21) Appl. No.: 17/375,934

(22) Filed: Jul. 14, 2021

Related U.S. Application Data
(60) Provisional application No. 63/052,010, filed on Jul. 15, 2020.

(51) Int. Cl. A61B 42/20

(52) U.S. Cl. CPC A61B 42/20

(57) ABSTRACT

A surgical sleeve for providing grip is provided. The surgical sleeve comprises a tubular gauze comprising an elongate body having a bore extending from a first end to a second end. The body has an opening at the first end and is closed at the second end. The surgical sleeve also includes a securing member operable to secure the opening to an appendage. The securing member is movable from an expanded diameter to a non-expanded diameter for insertion or removal of an appendage or instrument into or from the sleeve. The surgical sleeve also comprises a radiopaque thread secured to the body.

### CROSS REFERENCE TO RELATED APPLICATIONS

This application claims the benefit of U.S. Provisional Application No. 63/052,010, filed on Jul. 15, 2020, and entitled “Surgical Sleeve”, which is incorporated herein by reference in its entirety.

### FIELD

The present disclosure relates generally to surgical sleeves for securing gauze to an appendage. In various embodiments, surgical sleeves of the present disclosure comprise a tubular gauze and a securing member for securing an opening of the tubular gauze to an appendage.

### BACKGROUND

Surgical gloves are often formed from a stretchable, rubber (e.g., latex) material that may be or may become slippery during use. During surgery, wet tissues or surfaces may be difficult to grasp by a gloved finger or instrument. For example, retracting a thyroid gland during a thyroidectomy or a parathyroidectomy may be difficult as the thyroid may be too slick for a rubberized finger to grasp.

### SUMMARY

There is an unmet need to provide a surgical sleeve that improves grip and traction and that is securable to an appendage for use during surgery. Accordingly, embodiments of the present disclosure contemplate and provide a surgical sleeve for gripping and use during surgery. In various embodiments, a surgical sleeve for gripping is provided. In various embodiments, a tubular gauze has a bore extending from a first end to a second end. The tubular gauze has an opening at the first end and is closed at the second end. In various embodiments, the sleeve also comprises a fastener extending around the opening and operable to secure the opening to an appendage. The fastener is extendable from a first diameter to a second diameter when the fastener receives a force to stretch the fastener. The first diameter is less than the second diameter. In some embodiments, the fastener is at least one of an expandable ring, a zip-tie, a hook and loop strap, a cable tie, a clamp, and a pull tie. In various embodiments, the fastener is biased to the first diameter. In some embodiments, the fastener is an elastic band.

In various embodiments, the sleeve also comprises a radiopaque thread secured to the tubular gauze for detection by an x-ray. In various embodiments, the tubular gauze receives an appendage and the tubular gauze is secured to the appendage when the opening is secured to the appendage by the fastener. Embodiments of the present disclosure also comprise the fastener being an elastic band for securing the opening to the appendage. Embodiments of the present disclosure also comprise a pull tab positioned proximate the fastener and operable to receive the force to extend the fastener from the first diameter to the second diameter. Embodiments of the present disclosure also comprise the tubular gauze being elastic and operable to conform to the appendage. Embodiments of the present disclosure also comprise the first diameter being less than an appendage diameter.

In various embodiments, a surgical sleeve for gripping comprises a tubular gauze comprising an elongate body having a bore extending from a first end to a second end is provided. The gauze may be a thin, flexible fabric and/or a mesh fabric. In other embodiments, the surgical sleeve may be any material or fabric or a combination of any materials or fabrics including, but not limited to, cotton, silk, muslin, or the like. In some embodiments, the surgical sleeve is an elastic fabric that conforms to an appendage or instrument.

The body has an opening at the first end and is closed at the second end. In various embodiments, the sleeve comprises a securing member operable to secure the opening to an appendage. The securing member is movable from an expanded diameter to a non-expanded diameter and the expanded diameter is greater than the non-expanded diameter. In various embodiments, the surgical sleeve comprises a radiopaque thread secured to the elongate body for detection by an x-ray. Embodiments of the present disclosure also comprise the securing member for securing the opening to the appendage being at least one of an elastic band, an expandable ring, a zip-tie, a hook and loop strap, a cable tie, a clamp, and a pull tie.

A surgical finger cot according to at least one embodiment is provided comprising a gauze sleeve forming a cylinder extending and tapering from a first end to a second end. In some embodiments, the gauze sleeve is elastic and operable to conform to an appendage. The cylinder has an opening at the first end and is closed at the second end. The second end is rounded. In various embodiments, the cot comprises a fastener extending around the opening and operable to secure the opening to an appendage. The fastener is resilient and extendable from a first diameter to a second diameter when the fastener receives a force to stretch the fastener. In some embodiments, the fastener is an elastic band. The first diameter is less than the second diameter, and the fastener returns to the first diameter when the force is released. In various embodiments, the opening receives a force to extend the opening to the second diameter for the opening to receive the appendage and the force is released to retract the opening to the first diameter when the appendage meets the second end.

In various embodiments, the surgical finger cot further comprises a radiopaque thread secured to the gauze sleeve. In various embodiments, the surgical finger cot further comprises a pull tab positioned proximate the fastener, the pull tab operable to receive the force to extend the fastener from the first diameter to the second diameter.

A surgical sleeve according to at least one embodiments is provided. The surgical sleeve comprises a body having a cavity and an opening in communication with the cavity. In various embodiments, the body comprises a gauze sleeve. In some embodiments, the gauze sleeve is elastic and operable to conform to an object. In various embodiments, the body extends from a first end to a second end and the opening is disposed at the first end and the second end is closed. In various embodiments, the cavity is shaped to receive at least one of an appendage, an instrument, and a tool. In various embodiments, the body is cylindrically shaped.

The surgical sleeve also comprises a fastener extending around the opening and operable to secure the opening to an object, wherein the fastener is movable between a first diameter and a second diameter and is biased towards the first diameter; and a radiopaque thread secured to the body. In various embodiments, the fastener is an elastic band. In some embodiments, the opening receives a force to extend the opening to the second diameter for the opening to receive an object and the force is released to retract the opening to the first diameter when the object is in the cavity. The surgical sleeve, in some embodiments, further comprises a pull tab positioned proximate the fastener and operable to receive a force to extend the fastener from the first diameter to the second diameter.

This Summary is neither intended nor should it be construed as being representative of the full extent and scope of the surgical sleeve of the present disclosure. The present disclosure is set forth in various levels of detail in the Summary as well as in the attached drawings and the Detailed Description and no limitation as to the scope of the present invention is intended by either the inclusion or non-inclusion of elements or components. Additional aspects of the present invention will become more readily apparent from the Detailed Description, particularly when taken together with the drawings.

The above-described embodiments, objectives, and configurations are neither complete nor exhaustive. As will be appreciated, other embodiments of the invention are possible using, alone or in combination, one or more of the features set forth above or described in detail below.

The phrases “at least one,” “one or more,” and “and/or,” as used herein, are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together.

The term “a” or “an” entity, as used herein, refers to one or more of that entity. As such, the terms “a” (or “an”), “one or more,” and “at least one” can be used interchangeably herein. The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

### BRIEF DESCRIPTION OF THE DRAWINGS

The accompanying drawings, which are incorporated in and constitute a part of the specification, illustrate embodiments of the invention and together with the Summary given above and the Detailed Description of the drawings given below, serve to explain the principles of these embodiments. In certain instances, details that are not necessary for an understanding of the invention or that render other details difficult to perceive may have been omitted. It should be understood, of course, that the invention is not necessarily limited to the particular embodiments illustrated herein. Additionally, it should be understood that the drawings are not necessarily to scale.

FIG. 1 is a front perspective view of a surgical sleeve according to one embodiment of the present disclosure.

FIG. 2A is a front perspective view of the surgical sleeve of the embodiment of FIG. 1 positioned and secured to an appendage.

FIG. 2B is a rear perspective view of the surgical sleeve of the embodiment of FIG. 1 positioned and secured to the appendage of FIG. 2A.

Similar components and/or features may have the same reference label. Further, various components of the same type may be distinguished by following the reference label by a letter that distinguishes among the similar components. If only the first reference label is used, the description is applicable to any one of the similar components having the same first reference label irrespective of the second reference label.

### DETAILED DESCRIPTION

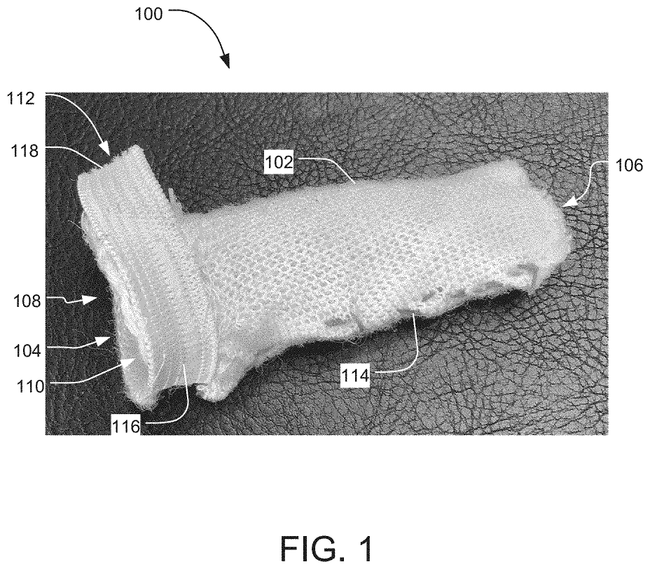

FIG. 1 is front perspective view of a surgical sleeve 100 according to one embodiment of the present disclosure. In the illustrated embodiment, the surgical sleeve 100 is a finger cot or finger sleeve. In alternative embodiments, the surgical sleeve 100 is a full glove or a partial glove. For example, the surgical sleeve 100 in some embodiments is a full glove for covering a user's hand and includes multiple finger sleeves and a covering for a palm and a back of the hand. In other embodiments, the surgical sleeve 100 includes one or more finger sleeves coupled to a covering for the palm, wrist, and/or back of the hand. In some embodiments, the sleeve 100 is provided in one size. In still other embodiments, the surgical sleeve 100 is shaped to fit over at least part of an instrument or tool. For example, the surgical sleeve 100, in some embodiments, is sized to cover a handle of a knife. The instrument may include, but is not limited to, a retraction device, a knife, a screw, a drill, a port, or a rod. In other embodiments, the surgical sleeve 100 is provided in multiple sizes to fit different hand and/or finger shapes and size and/or to fit instruments of different shapes and sizes. The surgical sleeve 100 can also vary in length and diameter.

In the illustrated embodiment, the surgical sleeve 100 comprises a body 102 that extends from a first end 104 to a second end 106. The body 102 includes an opening 108 at the first end 104 and is closed at the second end 106. The opening 108 opens into an interior or a cavity 110 of the body 102. In some embodiments, the interior 110 is a bore. In some embodiments, the body 102 is formed from gauze. In embodiments where the surgical sleeve 100 is a glove, at least a portion of the glove is formed from gauze. In some embodiments, the entire glove is formed from gauze. The gauze is sterile for use during surgery and provides traction or grip for handling, grasping, or holding objects that may be slippery. The gauze is formed from a flexible fabric. In some embodiments, the gauze is elastic and able to accommodate or conform to an appendage or instrument of various sizes when the appendage or the instrument is received in the interior 110. In other embodiments, the surgical sleeve 100 may be any material or a combination of materials including, but not limited to, cotton, silk, muslin, or the like.

In some embodiments, the body 102 is cylindrically or tubular shaped from the first end 104 to the second end 106. In other embodiments, the body 102 is cylindrically shaped at the first end 104 and tapers towards the second end 106. In some embodiments, the body 102 is formed in the shape of a finger cot or a finger cotyledon. In the illustrated example, the second end 106 is be rounded and shaped to receive a finger-tip. In other embodiments, the second end 106 may be any shape including, but not limited to, straight, angular, square, triangular, or the like.

The body 102 is formed by shaping (whether by cutting, tearing, or otherwise) a gauze sheet into a desired shape (e.g., tubular, tapered, cylindrical, etc.) and attaching edges of the sheet together as needed. In some embodiments, the edges are sewn together. In other embodiments, the edges are attached together by adhesion, stapling, riveting, or the like. In further embodiments, the gauze may be manufactured and sold in the desired shape.

In the illustrated embodiment, the surgical sleeve 100 includes a securing member 112 operable to secure the opening 108 to an object when the object is received in the interior 110, thereby securing the surgical sleeve 100 to the object. In some embodiments, the object is an appendage (e.g., a finger). In alternative embodiments, the object is a surgical instrument. The securing member 112 ensures that the surgical sleeve 100 remains on the appendage or the surgical instrument during a surgical procedure. For example, the securing member 112 will prevent the surgical sleeve 100 from being pulled off of the appendage or the instrument in applications where the appendage or the instrument is snagged or where the appendage or instrument is being removed from an interior portion of a patient. In embodiments where the surgical sleeve 100 is a glove, the securing member 112 is operable to secure an opening of the glove to a wrist of a user. For example, in some embodiments the securing member 112 is an elastic band operable to secure the opening of the glove to the wrist of the user.

In the illustrated embodiment, the surgical sleeve 100 includes one securing member 112. In other embodiments, the surgical sleeve 100 includes more than one securing member 112 for securing the sleeve 100 at multiple portions of the appendage or instrument. For example, the surgical sleeve 100 may have a securing member 112 at the opening 110 and another securing member 112 at a mid-portion of the body 102. In the illustrated embodiment, the securing member 112 is a fastener 116, such as an elastic band, extending around the opening 108. In embodiments where the sleeve 100 includes more than one securing member 112, each securing member 112 may be a different type of fastener 116 or each securing member 112 may be the same kind of fastener 116.

In the illustrated embodiment, the securing member 112 is wrapped around and secured to the appendage or instrument. The securing member 112 is expandable or movable between a first diameter and a second diameter. In embodiments where the first diameter is less than the second diameter, the first diameter is referred to as a non-expanded diameter and the second diameter is referred to as an expanded diameter. In such embodiments, the securing member 112 is movable to an expanded diameter to receive an appendage or instrument that has a diameter larger than the non-expanded diameter, and is moveable to the non-expanded diameter after the appendage or instrument is received in the interior 110. The non-expanded diameter is substantially equal to or less than a diameter of the appendage or instrument such that the securing member 112 conforms to and secures to the appendage or instrument when the securing member 112 is moved to the non-expanded diameter. In some embodiments, the securing member 112 is resilient and moves to the expanded diameter when the securing member 112 receives a force to pull or stretch the securing member 112 and returns to the non-expanded diameter when the force is released. For example, in some embodiments the securing member 112 is an elastic band that is stretched to the expanded diameter to provide enough room for an appendage or instrument to pass through the opening, and the elastic band is released when the appendage or instrument is positioned in the interior. When the elastic band is released, the elastic band returns to the non-expanded diameter. In other embodiments, the securing member 112 is static and remains in the expanded diameter or the non-expanded diameter when positioned in the expanded diameter or the non-expanded diameter.

In embodiments where the first diameter is greater than the second diameter, the appendage or instrument is received in the interior when the securing member 112 is in the first diameter. The securing member 112 is then movable to the second diameter after the appendage is received in the interior. The second diameter is substantially equal to or less than a diameter of the appendage or instrument such that the securing member 112 conforms to and secures to the appendage or instrument when moved to the second diameter.

In some embodiments, the securing member 112 is an elastic band sewn into or otherwise embedded or attached to a perimeter of the opening 110. In other embodiments, the securing member 112 is a string (e.g., rope, twine, or paracord) that is wrapped around the appendage or instrument and tied in a knot to secure the opening 110 to the appendage or instrument. In some embodiments, the securing member 112 is a strap having a free end which loops through and around a buckle or grommet and fastens to the strap, the buckle, or the grommet. In other embodiments, the securing member 112 is a strap having a free end which loops through and is secured by a double D-ring. In some embodiments, the securing member 112 is a bendable metal wire that is wrapped around the appendage or instrument to secure the opening 110 to the appendage or instrument. Other examples of such securing member 112 includes, but is not limited to, a zip tie, tape, a twist tie, an expandable ring, a cable tie, a clamp, a hose clamp, a pull tie, or a hook and loop strap commonly known as a Velcro® strap.

It will be appreciated that in some embodiments, the opening 110 itself is movable between a first diameter and a second diameter (whether because the body 102 is formed from an elastic material, or otherwise) and the securing member 112 may simply secure the opening 110 at the first diameter and/or the second diameter. For example, the securing member 112 may be a clasp having a first clasp member at a first location on the body 102 and near the opening 110 and a second clasp member at a second location on the body 102, near the opening and spaced from the first location. When the first clasp member is secured to the second clasp member, the opening 110 is secured or locked in the first diameter.

The surgical sleeve 100 also includes a radiopaque thread 114. In some embodiments, the surgical sleeve 100 does not include the radiopaque thread 114. The radiopaque thread 114 is visible in an X-ray image and, in some applications, provides a visual indicator of a position of the surgical sleeve 100 in image(s) taken during a surgical procedure. Such indicator may be helpful for tracking and/or positioning an instrument, for example. In the illustrated embodiment, the radiopaque thread 114 is sewn into the body 102. In other examples, the radiopaque thread 114 is adhered, stapled, or otherwise affixed to the body 102. In some embodiments, the radiopaque thread 114 is used to sew fasteners of the gauze together to form the body 102.

In the illustrated example, the surgical sleeve 100 also includes a pull tab 118. In some embodiments, the surgical sleeve 100 does not include the pull tab 118. The pull tab 118 provides a tab for gripping by a user to ease insertion or removal of the appendage or instrument into and from the surgical sleeve 100. More specifically, the pull tab 118 is operable to receive a force to move the securing member 112 between the first or non-expanded diameter and the second or expanded diameter. The pull tab 118 is positioned proximate the fastener or opening 108.

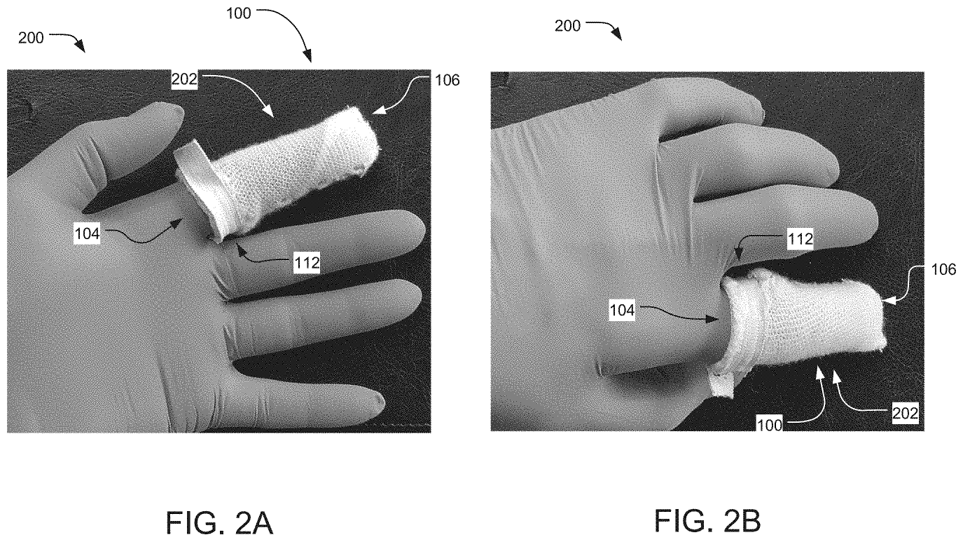

Turning to FIGS. 2A and 2B, a front perspective view of the surgical sleeve 100 positioned on and secured to an appendage 202 of a hand 200 and a rear perspective view of the surgical sleeve 100 positioned on and secured to the appendage 202 are respectively shown. As previously described, the surgical sleeve 100 may be used with any object, such as a surgical instrument. In the illustrated embodiment, the appendage 202 is a finger. As shown, the surgical sleeve 100 covers the entire finger, including the finger-tip. In other embodiments, the surgical sleeve 100 covers a portion of the finger less than the entire finger. During use, the surgical sleeve 100 provide grip to the finger such that the finger can grasp, grab, or grip a surface or object that may be slippery or slick.

In the illustrated embodiment, the surgical sleeve 100 is placed over a surgical glove. This provides for easy and quick use during surgery, as the surgeon can simply position and secure the surgical sleeve 100 over the glove during the operation without having to remove the glove or perform any extra procedures. The surgeon can also easily remove and/or replace the surgical sleeve 100 without having to remove the glove.

Although the following text sets forth a detailed description of numerous different embodiments, it should be understood that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims. To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning.

While various embodiments of the present invention have been described in detail, it is apparent that modifications and alterations of those embodiments will occur to those skilled in the art. Moreover, references made herein to “the present invention” or aspects thereof should be understood to mean certain embodiments of the present invention and should not necessarily be construed as limiting all embodiments to a particular description. It is to be expressly understood that such modifications and alterations are within the scope and spirit of the present invention.

What is claimed is:

1. A surgical sleeve for gripping comprising:
a tubular gauze having a bore extending from a first end to a second end, the tubular gauze having an opening at the first end and closed at the second end;
a fastener extending around the opening and operable to secure the opening to an appendage, the fastener being extendable from a first diameter to a second diameter when the fastener receives a force to stretch the fastener, the first diameter being less than the second diameter; and
a radiopaque thread secured to the tubular gauze for detection by an x-ray,
wherein the tubular gauze receives the appendage and the tubular gauze is secured to the appendage when the opening is secured to the appendage by the fastener.

2. The surgical sleeve of claim 1, further comprising a pull tab positioned proximate to the fastener, the pull tab operable to receive a force to extend the fastener from the first diameter to the second diameter.

3. The surgical sleeve of claim 1, wherein the tubular gauze is elastic and operable to conform to the appendage.

4. The surgical sleeve of claim 1, wherein the first diameter is less than a diameter of the appendage.

5. The surgical sleeve of claim 1, wherein the fastener is biased to the first diameter.

6. The surgical sleeve of claim 5, wherein the fastener is an elastic band.

7. The surgical sleeve of claim 1, wherein the fastener is at least one of an expandable ring, a zip-tie, a hook and loop strap, a cable tie, a clamp, and a pull tie.

8. A surgical finger cot comprising:
a gauze sleeve forming a cylinder extending and tapering from a first end to a second end, the cylinder having an opening at the first end and closed at the second end, the second end being rounded; and
a fastener extending around the opening and operable to secure the opening to an appendage, the fastener being resilient and extendable from a first diameter to a second diameter when the fastener receives a force to stretch the fastener, the first diameter being less than the second diameter, and the fastener returning to the first diameter when the force is released,
wherein the opening receives a force to extend the opening to the second diameter for the opening to receive the appendage and the force is released to retract the opening to the first diameter when the appendage meets the second end.

9. The surgical finger cot of claim 8, further comprising a radiopaque thread secured to the gauze sleeve.

10. The surgical finger cot of claim 8, further comprising a pull tab positioned proximate to the fastener, the pull tab operable to receive the force to extend the fastener from the first diameter to the second diameter.

11. The surgical finger cot of claim 8, wherein the fastener is an elastic band.

12. The surgical finger cot of claim 8, wherein the gauze sleeve is elastic and operable to conform to the appendage.

13. A surgical sleeve comprising:
a body having a cavity and an opening in communication with the cavity;
a fastener extending around the opening and operable to secure the opening to an object, wherein the fastener is movable between a first diameter and a second diameter and is biased towards the first diameter; and
a radiopaque thread secured to the body,
wherein the opening receives a force to extend the opening to the second diameter for the opening to receive an object and the force is released to retract the opening to the first diameter when the object is in the cavity.

14. The surgical sleeve of claim 13, further comprising a pull tab positioned proximate the fastener, the pull tab operable to receive the force to extend the fastener from the first diameter to the second diameter.

15. The surgical sleeve of claim 13, wherein the fastener is an elastic band.

16. The surgical sleeve of claim 13, wherein the body comprises a gauze sleeve.

17. The surgical sleeve of claim 16, wherein the gauze sleeve is elastic and operable to conform to an object.

18. The surgical sleeve of claim 13, wherein the body extends from a first end to a second end, wherein the opening is disposed at the first end, and the second end is closed.

19. The surgical sleeve of claim 13, wherein the cavity is shaped to receive at least one of an appendage, an instrument, and a tool.

20. The surgical sleeve of claim 13, wherein the body is cylindrically shaped.
